(19) United States
(12) Patent Application Publication
(10) Pub. No.: US 2002/0033179 A1
(43) Pub. Date: Mar. 21, 2002

(54) Adjustable penile constriction device

(76) Inventor: Pacifico Regalado Burgos, Glendale, CA (US)

Correspondence Address: PACIFICO R. BURGOS, Suite 6, 4253 Eagle Rock Bl., Los Angeles, CA, 90065, US

(21) Appl. No.: 09/920,878

(22) Filed: Aug. 2, 2001

Related U.S. Application Data
(60) Provisional application No. 60/222,982, filed on Aug. 4, 2000.

(51) Int. Cl. A61F 6/02

(52) U.S. Cl. 128/842

(57) ABSTRACT

The present invention is an adjustable penile constriction device for relieving penile venous leakage during erection, comprising of a main part that consists of a length of solid yet resilient material such as soft plastic, rubber and the like, and having ends with formations that interlock with similar formations on the surface of holes carried by a secondary part of the same material, to form an adjustable loop adaptable to and for effecting variable constriction around the human penile base and wherein, the formations on the ends of the said main part consist of alternating projections and recesses upon which the similar formations on the inner surface of the holes carried by the secondary part interlock with to allow the movement of the secondary part along the ends of the main part for securing the device unto the penile base, for adjusting its constrictive effect and for permitting its easy release from the penile base.

### CROSS-REFERENCE TO RELATED APPLICATION

This application covers the invention disclosed in my Provisional patent application Ser. No. 60/222,982 filed on Aug. 4, 2000.

### FIELD OF INVENTION

This invention relates to penile constriction devices and is particularly directed to erection enhancing-penile constriction devices which are adjustable to enable the user to readily vary the degree of penile constriction for a comfortable use and for easy release of the device from the penile base.

### BACKGROUND OF THE INVENTION

Among men, penile venous leakage is very common. This erectile condition is experienced by those who are able to generate an erection but could not maintain the quality of erection for a satisfactory intimate interaction.

Penile venous leakage occurs when the rate and amount of blood that gets into the penis during erection is not sufficient to create the pressure that is enough to compress the veins within the penile organ. When this happens, the penile veins are able to drain the blood back to the body faster than the penile arteries could bring blood into the penis. This results to smaller sized, less rigid and short-lived erection.

Penile venous leakage is highly prevalent among men with health disorders such as impotence, diabetes, prostate ailments and many other blood circulatory problems. Smokers, alcohol drinkers and overweight men are very likely to encounter this sexual problem.

Men with the best health, of any age and with normal erection, may also encounter Penile Venous Leakage problem. This is because the rate and volume of blood that gets into the penis during erection depend on the level of sexual arousal. The more sexually aroused the person gets, the faster and more amount of blood would normally flow into his penis for a good erection.

Unfortunately, sexual arousal is easily affected by stress, anxiety, physical fatigue or by simply not being very interested to have sex. In any of these cases, blood flow into the penis during erection will be slow and Penile Venous Leakage is very likely to occur.

Numerous devices for treating penile venous leakage are known to art. Among these devices are constriction rings that are usually made of elastic rubber material. These devices are placed around the penile base to create a constrictive effect enough to impede the outflow of blood from the penis via the penile veins that are primarily located on the penile surface.

By nature, the human penile organ however, come in a very wide variety of size. Because they rely upon elasticity to provide the necessary constriction, constriction rings are often made with very small diameter. Therefore, when positioned on the penile base, these devices are extremely painful to the user. Further, these rings do not allow for easy release and thus, forces the user to wait until his penis get limp before he is freed of the painful constriction.

There is therefore, a need for a better device that will enable the user to effectively relieve penile venous leakage and that which is easy to use, adjustable and easily releasable to provide for the user's comfort.

### BRIEF SUMMARY OF THE INVENTION

The principal objective of the present invention is to provide a better yet inexpensive device that will enable the user to effectively and comfortably relieve penile venous leakage.

Accordingly, it is the specific objective of the invention to provide a device that will allow the user to optimally constrict the superficial veins around his penile base to restrict the backflow of blood from his penis to his body during erection.

It is also a further object of the invention to provide a device having a means for the user to easily adjust the constrictive pressure effected by said device for it to be comfortable during use.

It is also an object of the invention to provide a device having means for the user to easily adapt said device on and release the same from his penis.

These objectives of the present invention are attained by providing a device comprising of a main part, consisting of a length of solid yet resilient material such as soft plastic, rubber and the like, having ends with formations that interlock with similar formations on the inner surfaces of holes carried by a secondary part that is made of material identical to that of the main part, to form an adjustable loop that is adaptable to and can effect variable constriction around the human penile base.

The aforesaid objectives and embodiments of the present invention will be apparent in the following detailed description, discussed with reference to the figures in the related drawings.

### BRIEF DESCRIPTION OF SEVERAL VIEWS OF THE DRAWINGS

FIG. 1 is the front view of the adjustable penile constriction device embodying the present invention;

FIG. 2 is the cross-sectional view of the present invention, showing the constriction adjustment-means of the device;

FIG. 3 is the top view of the main part of the present invention;

FIG. 4 is the side view of the main part of the present invention;

FIG. 5 is the side view of the secondary part of the invention;

FIG. 6 is the top view of the secondary part of the invention; and

FIG. 7 is the cross-sectional view of the secondary part of the invention.

### DETAILED DESCRIPTION OF THE INVENTION

The present invention is a device designed to create a constrictive effect on the superficial veins around the penile base to restrict the outflow of the blood from the penis during erection. This device is also intended to enable the user to easily adjust its constrictive effect and to provide for its quick release from the penis.

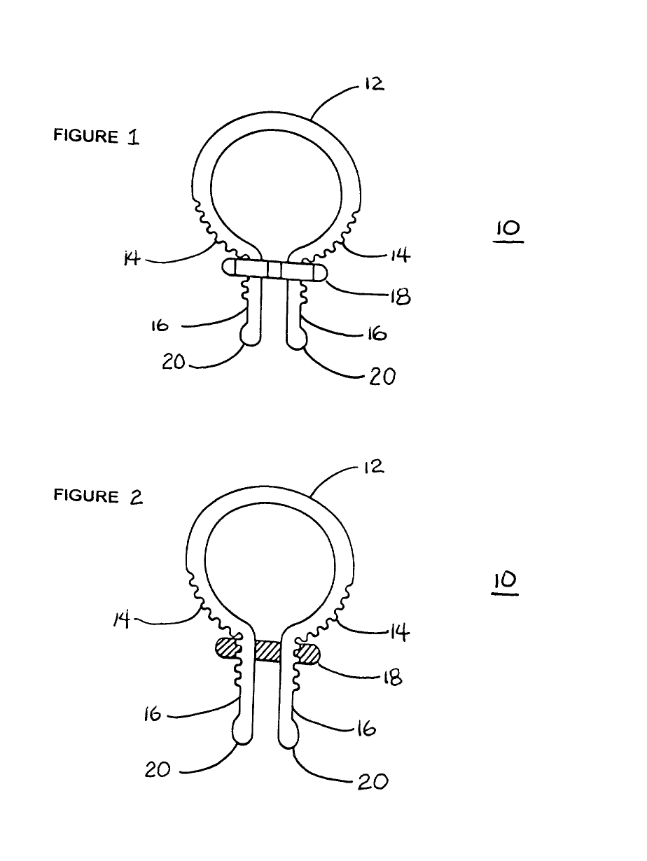

FIGS. 1 and 2 show the form of the present invention, which is taken for the purpose of illustration, of an adjustable penile constriction device, indicated generally at 10, is shown as comprising of a main part 12, consisting of a length of solid yet resilient material such as soft plastic, rubber and the like, whose ends pass through and interlock with holes carried by a secondary part 18 to form an adjustable and releasable loop for effecting variable constriction around the human penile base.

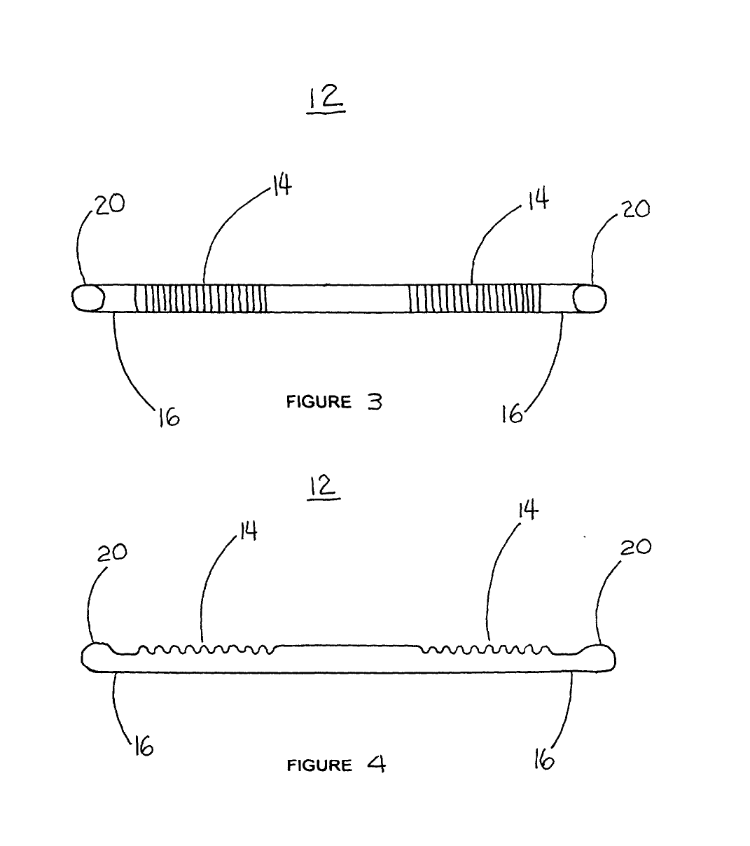

FIGS. 3 and 4 show separately the main part 12 of the present invention, consisting of a length of solid yet resilient material such as soft plastic, rubber and the like, having a desirably smooth surface and rounded corners, with alternating projections and recesses 14 formed on both ends 16 of the main part. The end tips of the main part may be enlarged 20 to facilitate grasping of said ends during use.

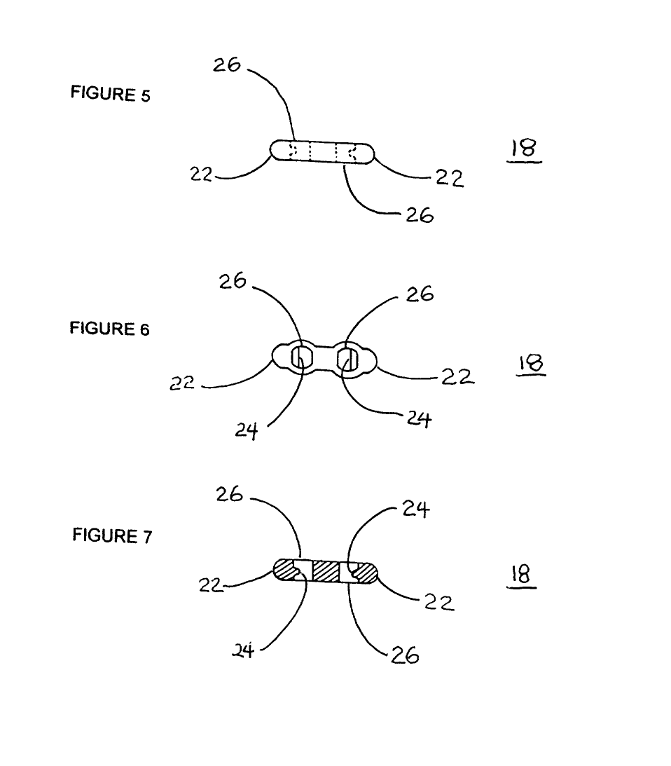

FIGS. 5, 6 and 7 show separately the secondary part 18 of the present invention, consisting of a piece of material identical to that of the main part 12. Said secondary part 18 is formed with a hole 26 on each of its end 22, through which the ends 16 of the main part 12 are passed through and interlock with the alternating projections and recesses 24 formed in the inner surface of the holes 26 carried by the secondary part 18, to form an adjustable loop 10 that is adapted on the penile base, not shown, for constricting the superficial penile veins during erection and thereby, relieving penile venous leakage.

In use, the user initially inserts one end 16 of the main part 12 through a hole 26 on the secondary part 18. He then, inserts the other end 16 of the main part 12 through the other hole 26 on the secondary part 18, with the alternating projections and recesses 14 on the ends 16 of the main part 12 interlocking with the same formations 24 in the inner surface of the holes 26 of the secondary part 18, to form the adjustable penile constriction loop 10.

The user slides the secondary part 18 towards the ends 16 of the main part 12 to increase the diametric size of the loop 10, for the device to easily slip through the penile head and shaft. The device 10 may be set around the penile base in front or behind the scrotum, not shown, before or during erection, with the secondary part 18 of the device placed on the dorsal side of the penile base.

The user slides the secondary part 18 upwards along the ends 16 of the main part 12 to decrease the diametric size of the device to effect and increase the constrictive pressure that the device will create around the penile base. The user slides the secondary part 18 downwards along the ends 16 of the main part 12 to increase the diametric size of the device to adjust and decrease the constrictive pressure on the penile base effected by the device 12 or to release it from the penis.

The main part 12 of the present invention may be provided in a plurality of lengths to accommodate the wide range in the diametric size of the human penis.

Obviously, numerous other variations and modifications can be made without departing from the spirit of the present invention, such as for the secondary part 18 to only carry one hole 26, instead of two as illustrated by the herein drawings, through which both ends 16 of the main part 12 pass through and interlock with.

Therefore, it should be clearly understood that the forms of the present invention as described herein and as shown in the figures of the accompanying drawings are illustrative only and are not intended to limit the scope of the present invention.

What I claim as my invention is:

1. An adjustable penile constriction device comprising:
a main part, consisting of a length of solid yet resilient material such as soft plastic, hard rubber and the like, having ends with formations that interlock with similar formations on the inner surface of holes carried by a secondary part made of material identical with that of the main part, to form an adjustable loop adaptable to and for effecting variable constriction around the human penile base.

2. The device of claim 1 wherein:
the formations on the ends of the said main part consist of alternating projections and recesses upon which similar formations of alternating projections and recesses on the inner surface of the holes carried by the secondary part interlock with, to allow the movement of the secondary part along the ends of the main part for securing the device on the penile base, for adjusting its constrictive effect and for its release.

3. The device of claim 1 wherein:
the said main part is desirably of smooth surface and rounded corners and formed with enlargements on its ends to facilitate grasping of said ends during use.

4. The device of claim 1 wherein:
the said main part is provided in a plurality of length sizes to accommodate the wide range of diametric sizes of the human penis.
